(19) United States
(12) Patent Application Publication
(10) Pub. No.: US 2017/0258319 A1
(43) Pub. Date: Sep. 14, 2017

(54) SYSTEM AND METHOD FOR ASSESSING EYESIGHT ACUITY AND HEARING ABILITY

(71) Applicant: KONINKLIJKE PHILIPS N.V., EINDHOVEN (NL)

(72) Inventors: Alina Weffers-albu, Boukoul (NL); Lukas Gorzelniak, Aachen (DE)

(21) Appl. No.: 15/529,271

(22) PCT Filed: Nov. 5, 2015
(86) PCT No.: PCT/EP2015/075852

§ 371 Date: May 24, 2017

(30) Foreign Application Priority Data
Nov. 27, 2014 (EP) 14195194.7

(51) Int. Cl. A61B 3/032; A61B 5/00; A61B 3/14; A61B 3/113; A61B 3/00; A61B 5/12; A61B 5/0496

(52) U.S. Cl. CPC A61B 3/032; A61B 5/123; A61B 5/4803; A61B 5/6898; A61B 5/0496; A61B 3/113; A61B 3/0025; A61B 3/145; A61B 2503/08; A61B 2505/07; A61B 2562/0257; A61B 2560/0247

(57) ABSTRACT

The present invention relates to a system (10) for assessing eyesight acuity of a subject (20), comprising: a display (12) for displaying graphics and/or text to the subject (20); an eye movement sensor (14, 14′) for monitoring an eye movement of the subject (20) while the subject (20) is watching the graphics and/or text displayed on the display (12); a processing unit (16) for assessing the eyesight acuity of the subject (20) based on an analysis of the monitored eye movement, wherein the analysis of the monitored eye movement includes an analysis of at least one of a duration of eye saccades, a frequency of eye saccades, a duration of eye fixations and a frequency of eye fixations in the monitored eye movement; and an output unit (18, 18′) for indicating the result of the assessment of the eyesight acuity. The present invention furthermore relates to a system (110) for assessing hearing ability of a subject (20).

### FIELD OF THE INVENTION

The present invention relates to a system for assessing eyesight acuity of a subject. The present invention also relates to a system for assessing hearing ability of a subject. Furthermore, the present invention relates to corresponding methods for assessing eyesight acuity and hearing ability of a subject. Still further, the present invention relates to computer programs for carrying out said methods.

### BACKGROUND OF THE INVENTION

The care of elderly people becomes more and more important as an aging of the population is a well-known trend in many countries. Especially due to the limited number of physicians and trained caregivers, the care of elderly people becomes a more and more challenging task. A lot of elderly people have to care themselves in many situations. Suitable equipment for self-care is thus of utmost importance.

Self-dependence and self-care activities of elderly people are critical for maintaining their state of health, well-being and quality of life in general. Vision and hearing ability are one of the essential abilities that enable self-care. An appropriate eyesight, for example, ensures that patients are able to read prescriptions of their medication. An appropriate hearing ability prevents patients from a lot of risks in their daily life.

Monitoring the trends in eyesight acuity and hearing ability of elderly people allows caregivers to become aware early in time when patients need support, thereby diminishing the risk of adverse events and occurrences caused by a lack of adherence to medication when patients become less able to read their prescriptions, to follow directions or to hear warning signals.

Monitoring the trends in eyesight acuity of elderly diabetic patients suffering from retinopathy is one of the tasks that become more and more important in view of the dramatic risks (of loosing ones eyesight) incurred when monitoring is not vigilant enough. “Retinopathy effects up to 80% of all patients who have had diabetes for ten years or more” (Kertes P J, Johnson T M, ed. (2007), “Evidence Based Eyecare”, Philadelphia, Pa. Lippincott, Williams and Wilkins, ISBN 0-7817-6964-7), while research indicates that “at least 90% of the new cases could be reduced if there was proper and vigilant monitoring and treatment of the eyes” (Tapp R J, Shaw J E, Harper C A et al. (June 2003), “The prevalence of and factors associated with diabetic retinopathy in the Australian population”, Diabetes Care 26(6): 1731-7. doi: 10.2337/diacare.26.6.1731.PMID 12766102).

Currently, eyesight ability and hearing ability are assessed in most of the cases based on an appointment by a professional who uses questionnaires and/or medical tests designed for those purposes. These assessments are only punctual and based on the patient's initiative. These rare and sporadic measurements of the patient ability, which cannot capture accurately the trends of eyesight and hearing ability, are often leading to a delay of support, as the moment when the patient starts deteriorating and thereby needing support is not captured. This has the following disadvantages: 1. Appointments with physicians are cumbersome for elderly patients with vision and hearing problems, as they already depend on others (e.g. for transportation). They require physical effort on the side of the patient, a fair amount of scheduling for all parties involved, and discomfort during waiting times involved at the physician. 2. Eyesight and hearing deterioration trends will likely not be caught early enough in time to be able to intervene sufficiently effective. 3. Caregivers are informed of the patient status only at the time of the appointment, which might be too late, leading to more intense and/or advanced interventions that often imply increased patient discomfort, cost more and have less likelihood of success. 4. Such an approach implies increased risk in the occurrence of adverse events (such as COPD or heart failure exacerbations) in all patients caused by a lack of adherence to medication when patients become less able to read their prescriptions.

There is thus a need for systems and devices which allow especially elderly patients to test their eyesight acuity and hearing ability themselves in the home environment.

### SUMMARY OF THE INVENTION

It is an object of the present invention to provide a system and method which allow especially elderly people to assess their eyesight acuity and hearing ability by themselves in their home environment. Such systems and methods shall allow a frequent monitoring and unobtrusive assessment of eyesight and hearing ability to detect deteriorations as early as possible. These systems and methods shall also support people in their everyday life.

In a first aspect of the present invention a system for assessing eyesight acuity of a subject is presented which comprises:

a display for displaying graphics and/or text to the subject;

an eye movement sensor for monitoring an eye movement of the subject while the subject is watching the graphics and/or text displayed on the display;

a processing unit for assessing the eyesight acuity of the subject based on an analysis of the monitored eye movement, wherein the analysis of the monitored eye movement includes an analysis of at least one of a duration of eye saccades, a frequency of eye saccades, a duration of eye fixations and a frequency of eye fixations in the monitored eye movement; and

an output unit for indicating the result of the assessment of the eyesight acuity.

In a further aspect of the present invention a method for assessing eyesight acuity of a subject is presented which comprises:

displaying graphics and/or text on a display;

monitoring an eye movement of the subject by means of an eye movement sensor while the subject is watching the graphics and/or text displayed on the display;

assessing the eyesight acuity of the subject based on an analysis of the monitored eye movement, wherein the analysis of the monitored eye movement includes an analysis of at least one of a duration of eye saccades, a frequency of eye saccades, a duration of eye fixations and a frequency of eye fixations in the monitored eye movement; and

indicating the result of the assessment of the eyesight acuity.

In a still further aspect of the present invention, a computer program for carrying out said method is presented.

Preferred embodiments of the invention are defined in the dependent claims. It shall be understood that the claimed method and the claimed computer program have similar and/or identical preferred embodiments as the claimed system and as defined in the dependent claims.

The presented system makes use of a display which displays graphics and/or text to the subject. While the subject is watching the graphics and/or reads the text displayed on the display, the system monitors the eye movement of the subject in order to determine characteristics of watching at the graphics and/or reading the text. The system may thus make an assessment with respect to ease of reading and may thereby determine the eyesight ability level of the subject. The processing unit receives as input the eye movement signal produced by the eye movement sensor, analyses this eye movement signal and detects the ease of reading based on this analysis. The processing unit is also configured to steer the display, such that information regarding size, colour and sharpness of the displayed graphics and/or text are also known parameters on which the assessment of the eyesight acuity of the subject may be based.

According to a preferred embodiment a known text is displayed on the display, since this allows analysing the ease of reading of the subject best. However, it is also possible to display graphics on the display, since this may eliminate other influences for reading difficulty, such as e.g. dyslexia of the subject. However, it shall be noted that a differentiation of reading faults caused by the quality of eyesight on the one hand and dyslexia on the other hand may be also made by observing the eye movements of the subject if the subject is reading a text. This will be explained further below in detail.

The components of the presented system may be arranged remote from each other. However, according to a preferred embodiment, the display, the eye movement sensor, the processing unit and the output unit are integrated in a single portable device. Such a single portable device may, for example, be realized as a tablet PC or a mobile smartphone which are equipped with the above-mentioned system components.

The output unit of the presented system is configured to indicate the results of the assessment of the eyesight acuity. This indication may be either given directly to the subject him-/herself in audible or visual form. The output unit may, for example, comprise a loudspeaker or may alternatively be comprised in the display, such that the result is presented to the subject in written form on the display. Alternatively, the results of the assessment of the eyesight acuity of the subject may be directly transferred to a physician by means of the output unit, e.g. via the Internet. In the latter-mentioned case the output unit comprises a data interface which is connected to a server, a network, or directly to the Internet. This enables informing a caregiver or physician with a recommendation for a consult of the subject as soon as a deterioration of the eyesight accuracy of the subject is determined.

The advantages of the presented system are as follows: 1. The presented solution offers an easy-to-use, low effort and unobtrusive way of monitoring eyesight ability. 2. The presented system may reduce the risk of significant retinopathy deterioration given the ability of frequent use of the system in the home environment of the subject. 3. Caregivers and physicians may be informed by the system as soon as a beginning deterioration trend is detected, allowing for prompt interventions that can effectively stop the deterioration. 4. Adverse events occurrence (due to diminished medication adherence) is very much diminished by the system.

In summary, the presented system proposes a proactive approach that enables detecting eyesight deterioration and preventing adverse events. As a consequence, the presented system may enable a reduction of hospitalizations and advanced, high-intensity medical treatments.

The analysis of the monitored eye movement performed by the processing unit includes an analysis of at least one of a duration of eye saccades in the monitored eye movement, a frequency of eye saccades in the monitored eye movement, a duration of eye fixations in the monitored eye movement and a frequency of eye fixations in the monitored eye movement.

The processing unit is, in other words, configured to analyse at least one of a duration of eye saccades, a frequency of eye saccades, a duration of eye fixations and a frequency of eye fixations in the eye movement signal produced by the eye movement sensor. An “eye saccade” is a fast forward or backward movement of the eyes. Smaller saccades typically occur while the eyes move over the words in a line of the displayed text. A short saccade is indicated by a rapid short distance eye movement to the right side. Large saccades may be typically observed when the eyes move back to the beginning of the next line of the displayed text. A long saccade is indicated by a rapid longer distance eye movement than a short saccade in the left-down direction. An “eye fixation” is a static state of the eyes during which gaze is held upon a specific location. Humans typically alternate saccadic eye movements and eye fixations. For that reason the term “duration of an eye fixation” can also be referred to as the time between two saccades during which the eyes are relatively stationary. An eye fixation is usually indicated by the maintained position of the eyes over a longer period of time.

Eye saccades and eye fixations may be determined by the processing unit by means of identifying signal peaks in the eye movement signal within the time domain. The processing unit may be particularly configured to calculate differences in signal amplitude between two adjacent peaks within the eye movement signal. Adjacent peaks with negligible amplitude differences may be identified as an eye fixation. The time span between the first and the last peak belonging to the fixation determines the fixation duration. Adjacent peaks with medium amplitude differences may be identified as short saccades. The time span between the first and the last peak belonging to a short saccade determines the short saccade duration. Adjacent peaks with significant amplitude differences may determine long saccades. The time span between the first and the last belonging to a long saccade determines the long saccade duration.

The processing unit may be configured to compare the signal peaks and the time distances between two adjacent signal peaks to predetermined threshold values in order to distinguish between fixations, short saccades and long saccades. These threshold values may be either predetermined based on average values of users in a public database or they may be derived from the eye movement signal itself in a type of learning mode. The analysis of eye fixations and eye saccades is particularly advantageous in case of displaying text on the display. As the subject experiences difficulty at reading, he/she will need to fixate longer and more often on words in order to determine the letters. This has implications in terms of the eye movement signal characteristics (fixations and saccades) as follows: An on-going deterioration of eye sight acuity of the subject is indicated by an increased trend of the frequency of fixations. Also an increased time duration of the fixations would indicate an on-going deterioration. A further indication of an on-going deterioration of the eyesight acuity is a slower saccadic movement. The reading ability level which is an indicator for the eyesight acuity of the subject may thus be assessed by the processing unit based on an analysis of a trend of occurring saccades and fixations of the eyes of the subject over time while the subject is reading the text displayed on the display.

According to an embodiment of the present invention, the eye movement sensor may comprise a camera. The eye movement signal is in this case an optical signal. One could, for example, take a video camera embedded in a tablet PC. However, external video cameras could be used as well. In case of using a video camera, the eye movement signal is configured to perform an eye/iris detection and/or gaze detection in order to monitor the eye movement of the subject.

In an alternative embodiment of the present invention, the eye movement sensor comprises an electrooculograph. Electrooculographic (EOG) sensors measure the corneo-retinal standing potential which exists between the front and the back of the human eye. The resulting signal is called electrooculogram. EOG sensors usually include a pair of electrodes or a plurality of electrodes which are adapted to be placed either above and below an eye or to the left and right of an eye. Instead of attaching the EOG sensors directly to the subject's head, the EOG sensors could be also embedded in an extra device (e.g. in glasses) which is attachable to the head of the subject.

According to a further embodiment, the processing unit is configured to control the display to vary at least one of a size, colour and sharpness of the graphics and/or the text over time, wherein the processing unit is configured to assess the eyesight acuity of the subject based on the analysis of the monitored eye movement and the variation of the at least one of the size, colour and sharpness of the graphics and/or the text over time.

It is particularly preferred that the processing unit is configured to control the display to gradually reduce the size of the graphics and/or the text over time while monitoring the eye movement of the subject. In this case the processing unit is able to determine the influence of the size reduction of the graphics and/or the text having onto the reading ability of the subject. The processing unit may be particularly configured to analyse the eye saccades and the eye fixations of the subject when reading the text which is continuously getting smaller. This type of analysis is specifically becoming more robust if the system is able to determine the distance between the display and an eye of the subject.

According to an embodiment of the present invention, the system further comprises a proximity sensor for measuring a distance between the display and an eye of the subject, wherein the processing unit is configured to assess the eyesight acuity of a subject based on the analysis of the monitored eye movement and the distance between the display and the eye of the subject. The system may then even better determine how the subject behaves when the size of the graphics and/or the text displayed on the display is varied, or when the colour and/or shortness of the displayed graphics and/or text are changed.

The distance between the eye of the subject and the display is not only an important parameter as it gives an indication regarding the subjective size feeling of the displayed graphics and/or text, but also as it allows distinguishing between different types of eye sight visibility. Patients having myopia will tend to bring the display closer to their eyes when the displayed graphics and/or text are decreased. Patients having astigmatism, hypermetropia or presbyopia will on the other hand tend to position the display farther away from their eyes when the displayed graphics and/or text are decreased.

Measuring the distance between the display and the eyes of the subject also enables distinguishing between eyesight disability and dyslexia. If only dyslexia impedes reading, the distance between the display and the eyes does not vary too much when the displayed graphics and/or text is decreased. On the other hand, dyslexia does not significantly change over time, while eyesight does. Hence, if reading ability worsens over time, it is in most of the cases because of the eyesight acuity worsens. The system may thus carry out a calibration phase to determine patient personal baseline parameter values during reading and thereby monitor deterioration by comparison with the baseline parameter values. This may be accomplished as follows: The system may observe the signal characteristics of the eye movement signal over a plurality of sessions in order to determine a “normal”, personal reading speed of the subject. From thereon the system may start to monitor both parameters and determine whether these parameters change over time. This will be explained further below in detail.

According to a further embodiment, the system may further comprise a microphone for recording a voice of the subject while reading out loud the graphics and/or the text displayed on the display, wherein the processing unit is configured to perform a speech recognition, and wherein the processing unit is configured to assess the eyesight acuity of the subject based on the analysis of the monitored eye movement and a comparison of the speech recognition with the graphics and/or the text displayed on the display. This may further increase the robustness of the assessment. The processing unit may be particularly configured to compare the text which is determined from the speech recognition with the text actually displayed on the display in order to determine the accuracy of reading. Any difficulty of reading could be inferred from the speed of the user's speech. This speech signal may be compared with the eye movement signal in order to increase the robustness of detecting eyesight disabilities. Such an embodiment is also advantageous if graphics (without text) are displayed on the display. The processing unit may, for example, be configured to display graphics like arrows or circles with an opening on the display. The user then has to identify the direction of the arrows or the position of the opening of the circles and accordingly has to say: left, right, up, down, etc. This will be registered by the speech recognition performed in the processing unit in order to compare the recognized speech with the actually displayed graphics. The eye movement, display-to-eye distance and/or variation of the size, color and/or shape of the graphics may be included into the assessment of the eyesight acuity in almost the same manner as if text is displayed on the display.

According to a further embodiment, the processing unit may be configured to adjust at least one of (i) a size of the graphics and/or text displayed on the display, (ii) a luminosity of the display, and (iii) a contrast of the display based on the analysis of the monitored eye movement. This may be particularly done when the system is used for reading apart from assessing the eyesight acuity. The system may then adjust the text size, the luminosity and the contrast of the display to ease the reading process. As these parameters are adjusted, the ease of the reading may be assessed again to check whether the parameters have been adapted in the most optimal way given the patient disability. In other words, the system changes the settings in a personalized way for the patient.

According to a further embodiment, the system may further comprise an ambient light sensor for measuring a brightness value of light in the ambience of the system, wherein the processing unit is configured:

to generate a feedback regarding the measured brightness value of the ambient light, which feedback is indicated via the output unit; or

to adjust at least one of (i) a size of the graphics and/or text displayed on the display, (ii) a luminosity of the display, and (iii) a contrast of the display based on the measured brightness value of the ambient light, or

to incorporate the measured brightness value of the ambient light in the assessment of the eyesight acuity of the subject.

Measuring the brightness value of the ambient light may thus cause the following possible reactions of the system:

1. If the brightness level of the ambient light is detected to be below a certain threshold value, the system may give a feedback to the subject via the output unit. This feedback may be given in visual or audible form. The subject may, for example, then be instructd to change the ambient light level or to move to another location with more light. Incorrect assessments of the eyesight acuity may thus be prevented.

2. The processing unit may adjust the size of the graphics and/or the text, the luminosity of the display, and/or the contrast of the display based on the measured brightness value of the ambient light. This may help to provide for each assessment of the eyesight acuity almost the same surrounding parameters independent of the ambient light level.

3. The processing unit may account for the measured brightness value of the ambient light, i.e. correct the assessment of the eyesight acuity of the subject based upon the measured brightness value of the ambient light. This also allows preventing misinterpretations of the eyesight acuity assessment.

According to a further embodiment, the processing unit may be configured to output instructions for the subject via the output unit, wherein said instructions support the subject in using the system. These instructions may, for example, include instructions to the subject how to read the text displayed on the display and/or when to start reading. The subject may also be instructed to keep the display closer or farther away from his/her eyes. The instructions may also include instructions including the indication of the results of the assessment of the eyesight acuity and possible consequences. For example, the subject may be instructd to make an appointment with a physician. The system may also instruct the subject to perform an eyesight test in the above-mentioned way by generating visual or audible reminders in constant intervals, e.g. every day, every hour or once in a month. These reminders may, of course, be repeated before a scheduled test. If the subject does not perform a scheduled test, the system may also inform the caregiver, the physician or someone else of the family that the subject missed to take part of the test by means of the system.

According to a further embodiment, the system further comprises a storage unit for storing the result of the assessment of the eyesight acuity each time the eyesight acuity of the subject is assessed by the processing unit, wherein the processing unit is configured to compare each new assessment with former assessments stored in the storage unit in order to derive a trend of the eyesight acuity of the subject over time. Such trend derivations are particularly advantageous, since trends, e.g. deteriorations of eyesight acuity over time, are easier to detect than an absolute eyesight acuity assessed in only one assessment. By identifying trends, personal characteristics of the subject, e.g. being a slow or fast reader, are diminished, since the system only looks at the change of the speed of reading over time.

In a second aspect of the present invention, a system for assessing hearing ability of a subject is presented which comprises:

a loudspeaker for generating a sound;

a sound variation unit for varying a frequency and/or loudness of the sound;

a feedback unit for receiving feedback at what frequency and/or loudness the sound may be recognized by the subject;

a proximity sensor for measuring a distance between the loudspeaker and the subject;

a processing unit for assessing the hearing ability of the subject based on an analysis of the received feedback and the distance between the loudspeaker and the subject; and

an output unit for indicating the result of the assessment of the hearing ability.

In a still further aspect of the present invention, a method for assessing hearing ability of a subject is presented which comprises:

generating a sound;

varying a frequency and/or loudness of the sound;

receiving feedback at what frequency and/or loudness the sound may be recognized by the subject;

measuring a distance between the loudspeaker and the subject;

assessing the hearing ability of the subject based on an analysis of the received feedback and the distance between the loudspeaker and the subject; and

indicating the result of the assessment of the hearing ability.

Furthermore, a computer program for carrying out said method is presented.

Preferred embodiments of the system for assessing hearing ability of a subject are defined in dependent claims 16-18. It shall be understood that the claimed method and the claimed computer program have similar and/or identical preferred embodiments as the claimed system.

The presented system for assessing hearing ability is based on a similar idea as the system for assessing eyesight acuity explained above. The system for assessing hearing ability of the subject generates a sound via a loudspeaker, wherein the frequency and/or the loudness of the sound is varied over time. During this variation the user may give a feedback via a feedback unit to indicate at what frequency and/or loudness he/she recognizes the sound. At the same time, the proximity sensor measures the distance between the loudspeaker and the subject, i.e. the ears of the subject. The processing unit then assesses the hearing ability of the subject based on an analysis of the distance between the loudspeaker and the subject and the frequency and/or loudness that is indicated by the subject to be recognizable.

During the assessment the loudness of the sound is preferably constantly increased. Alternatively or additionally, the frequency of the sound may be decreased over time. This variation of the sound may be either done manually by the subject him/herself via a user interface, or the sound variation unit may be configured to automatically vary the frequency and/or loudness of the sound over time.

The feedback unit may comprise a simple button that may be pressed by the subject as soon as he/she recognizes the sound. Alternatively, the feedback unit may be integrated in a display which comprises a touchscreen. In this case, the subject simply has to touch the display as soon as he/she recognizes the sound. According to a further alternative, the feedback unit may comprise a microphone such that the subject may give the feedback in audible form.

The system for assessing hearing ability may have similar or even the same embodiments as the above-mentioned system for assessing eyesight acuity.

According to an embodiment, the system further comprises a microphone for measuring a sound level in the ambience of the system, wherein the processing unit is configured:

to generate a feedback regarding the measured ambient sound level, which feedback is indicated via the output unit; or

to adjust the frequency and/or loudness of the generated sound based on the measured ambient sound level, or

to incorporate the measured ambient sound level in the assessment of the hearing ability of the subject.

According to a further embodiment, the processing unit may be configured to output instructions for the subject via the output unit, wherein said instructions support the subject in using the system.

According to a still further embodiment, the system may further comprise a storage unit for storing the result of the assessment of the hearing ability each time the hearing ability of the subject is assessed by the processing unit, wherein the processing unit is configured to compare each new assessment with former assessments stored in the storage unit in order to derive a trend of the hearing ability of the subject over time.

Also similar as in the above-mentioned system for assessing eyesight acuity of the subject, the components of the system for assessing hearing ability of the subject may be realized as components which are remote from each other or integrated into a single device. According to a preferred embodiment, the loudspeaker, the sound variation unit, the feedback unit, the proximity sensor and the processing unit are integrated in a single portable device.

### BRIEF DESCRIPTION OF THE DRAWINGS

These and other aspects of the invention will be apparent from and elucidated with reference to the embodiments described hereinafter. In the following drawings

FIG. 1 shows a first embodiment of a system for assessing eyesight acuity according to the present invention, wherein FIG. 1A schematically shows a handling of the system according to the first embodiment, and wherein FIG. 1B schematically shows the components of the system according to the first embodiment;

FIG. 2 shows a second embodiment of the system for assessing eyesight acuity, wherein FIG. 2A shows a handling of the system according to the second embodiment, FIG. 2B shows the components of the system according to the second embodiment, and wherein FIG. 2C shows an alternative handling of the system according to the second embodiment;

FIG. 3 shows an example of an eye movement signal;

FIG. 4 schematically shows a method for assessing eyesight acuity according to the present invention;

FIG. 5 shows an embodiment of a system for assessing hearing ability, wherein FIG. 5A schematically shows a handling of said system, and wherein FIG. 5B schematically shows the components of said system; and

FIG. 6 schematically shows a method for assessing hearing ability according to the present invention.

### DETAILED DESCRIPTION OF THE INVENTION

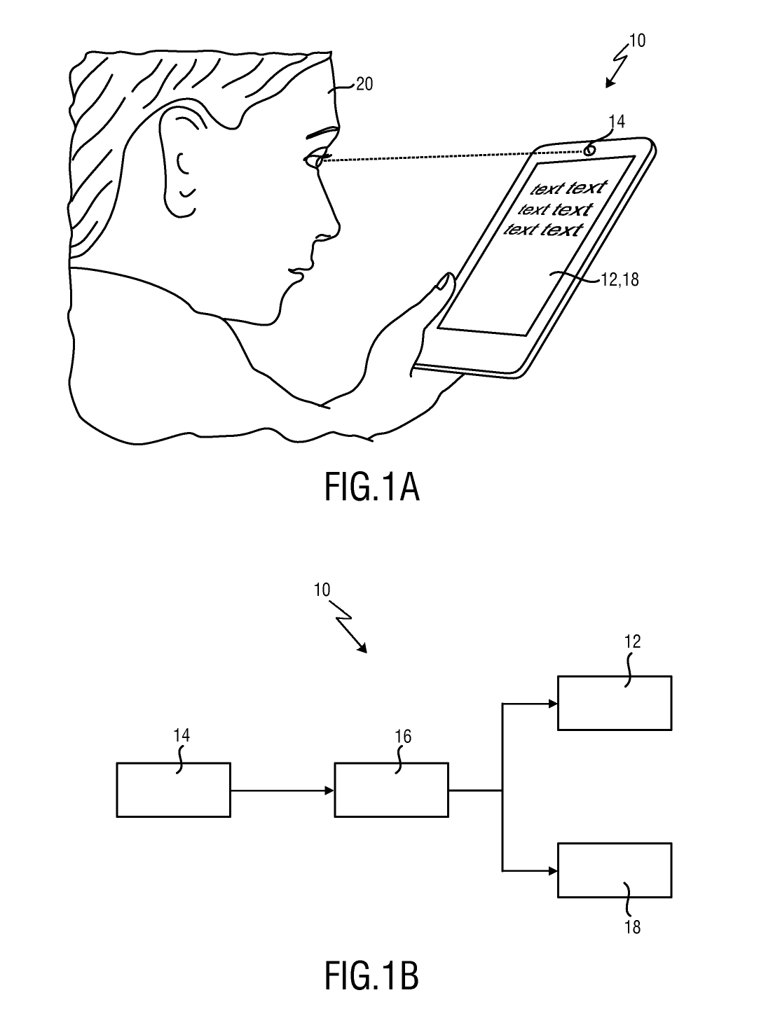

FIG. 1 shows a first embodiment of a system for assessing eyesight acuity according to the present invention. The system is therein in its entirety denoted with reference numeral 10. FIG. 1A schematically shows the handling of the system 10, and FIG. 1B shows the components of the system 10 according to the first embodiment.

In the shown example the system 10 is comprised in a tablet PC. However, the system 10 may also be comprised in a portable smartphone or in another portable computing device. The system 10 may also be comprised in a stationary desktop computing device.

The system 10 comprises a display 12, an eye movement sensor 14, a processing unit 16 and an output unit 18.

The display 12 is configured to display graphics and/or text to a subject 20. The display 12 may comprise any type of display means, such as e.g. a LCD display, a LED display or a plasma display. The display 12 may furthermore be operable to function as a touchscreen.

The eye movement sensor 14 is configured to monitor an eye movement of the subject 20 while the subject 20 is watching the graphics and/or reading the text displayed on the display 12. The eye movement sensor 14 according to the first embodiment comprises a digital camera, such as a digital video camera commonly used and embedded in tablet PC devices. The camera 14 preferably comprises an automatic focus unit which is configured to focus on the eyes of the subject 20.

The processing unit 16 is configured to assess the eyesight acuity of the subject 20 based on an analysis of the eye movement monitored by the camera 14. According to the first embodiment of the system 10, the processing unit 16 is preferably configured to perform a preprocessing of the camera signal in order to derive an eye movement signal which is indicative of the movement of the eyes of the subject 20. This is preferably done by means of an eye and/or iris detection during image processing, such that the gaze of the subject 20 is determined. The processing unit 16 may comprise a microchip or any other type of CPU having software stored thereon for carrying out the above-mentioned image and signal processing steps.

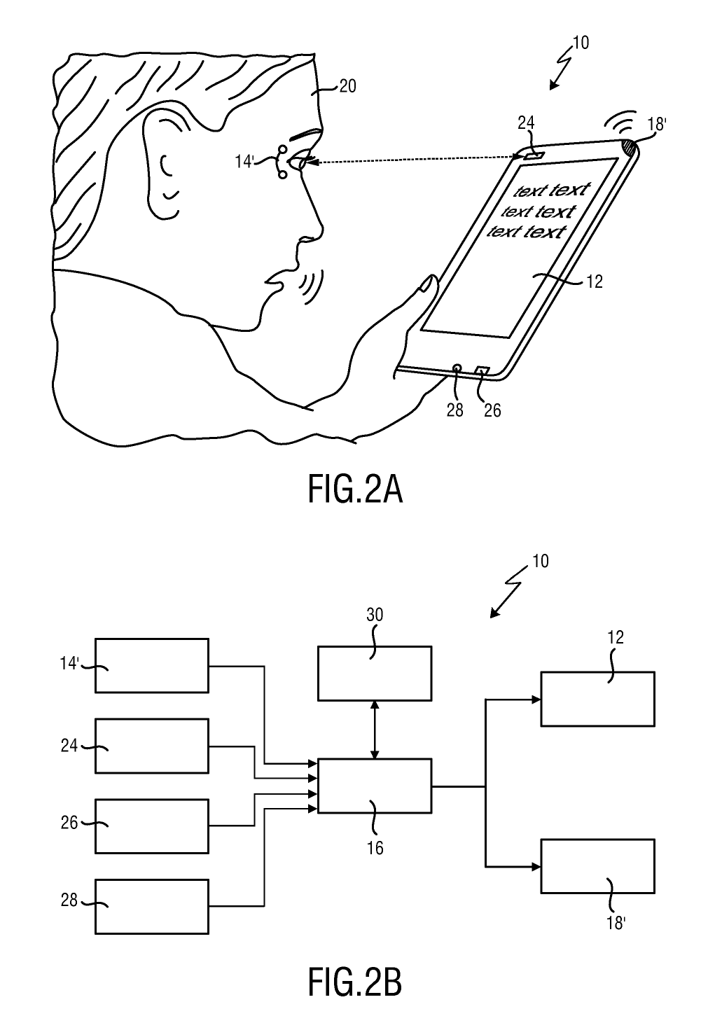

The output unit 18 is configured to indicate the results of the assessment of the eyesight acuity performed by the processing unit 16 to the subject 20. According to the first embodiment of the system 10, the output unit 18 may be a part of the display 12. The results of the eyesight acuity assessment may thus be fed back to the subject 20 in written form. Alternatively, it is also possible to realize the output unit 18 in the form of a loudspeaker, as this will be come apparent from the second embodiment shown in FIGS. 2A-2C. Additionally, the output unit 18 may comprise a data interface, such as a USB interface, a LAN interface or an Internet modem, such that the results of the eyesight acuity assessment may be transferred to a third party (e.g. a physician, a caregiver or a family member) via a network (e.g. the Internet).

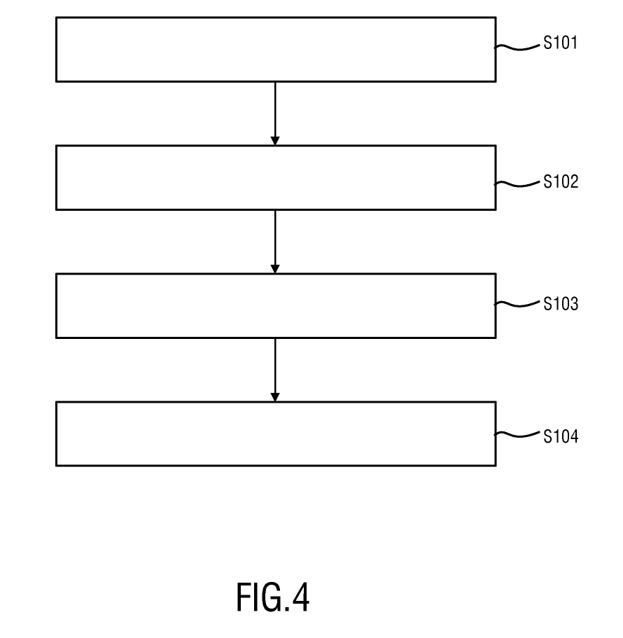

FIG. 4 schematically shows a method for assessing the eyesight acuity of the subject 20 according to the first embodiment. In a first step S101 the subject 20 is shown a text on the display 12. In a second step S102 the eye movement of the subject 20 is monitored by means of the eye movement sensor 14 while the subject 20 is reading the text on the display 12. An eye movement signal, which is indicative of the eye movement of the subject 20, is derived in the processing unit 16.

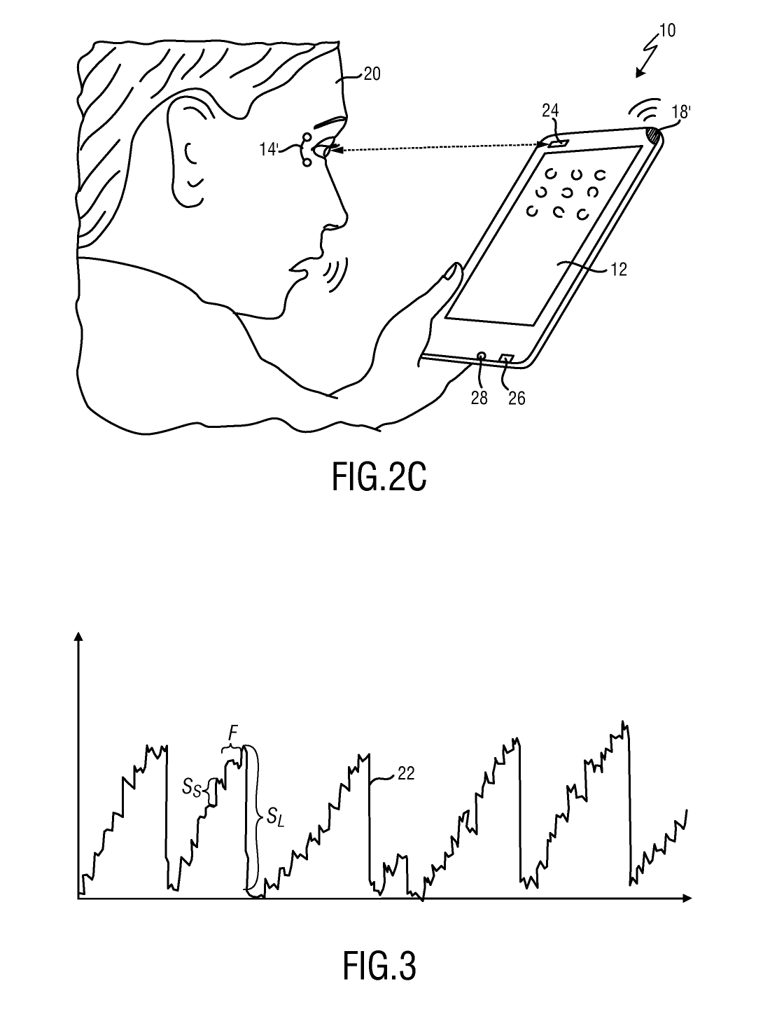

An example of such an eye movement signal 22 is shown in FIG. 3. Therein, the eye movement of the subject is plotted over time. The axis of ordinate in other words shows the distance of the eye movement, while the axis of abscissae shows the time.

In step S103 the eye movement signal 22 is analysed in order to assess the eyesight acuity of the subject 20. This is done in the processing unit 16. The processing unit 16 is preferably configured to analyse the eye movement signal 22 in the time domain. However, it is also possible to transfer the eye movement signal 22 into the frequency domain and to analyse it in the frequency domain.

The analysis of the eye movement signal 22 performed by the processing unit 16 preferably includes a detection and analysis of saccades and fixations of the subject's eyes. This may be done by means of an automatic detection of peaks within the eye movement signal 22. The processing unit 16 is configured to detect and calculate differences between two adjacent peaks in the eye movement signal amplitude. Adjacent peaks with almost negligible amplitude differences (smaller than a first threshold value), i.e. an approximately flat section of the eye movement signal 22, are indicative of an eye fixation F which results from the fact that the subject 20 maintains the position of the eyes over a comparatively longer period of time. Adjacent peaks with medium amplitude differences (larger than the first threshold value but smaller than a second threshold value) may be indicative of a short saccade S_{S} which may result from a rapid short distance eye movement from left to right. Adjacent peaks with a significant amplitude difference (larger than the second threshold value) may be indicative of a long saccade S_{L} which usually results from a rapid longer distance eye movement in the left-down direction. In other words, eye fixations F occur when the subject's eyes focus for a certain period of time onto one and the same text position or word in the text. Short saccades S_{S} occur when the subject 20 is reading along a text line and the eyes move within the line from left to right over or along the words in said text line. Longer saccades S_{L} occur when the subject 20 jumps with his/her eyes from an end of one text line to a beginning of the next text line.

The eyesight acuity of the subject 20 may be assessed by means of an analysis of a duration of the eye saccades detected in the eye movement signal 22, a frequency of the eye saccades detected in the eye movement signal 22, a duration of eye fixations detected in the eye movement signal 22 and/or a frequency of the eye fixations detected in the eye movement signal 22. An increasing trend of the frequency of eye fixations F may be an indication of an on-going deterioration of the eyesight acuity. Other indicators for an on-going deterioration of the eyesight acuity of the subject 20 are an increasing tendency of the fixation time durations or a slower saccadic movement of the eyes. The processing unit 16 is preferably configured to monitor such indicators over time (not only over time during one assessment/test, but also by comparing different test results with each other over longer periods of time, like a plurality of weeks or months). It is especially preferred that the size, colour and/or sharpness of the displayed text is decreased over time during each session/test in order to being able to monitor the influence of such a reduction/change onto the reading speed and reading behavior. This enables conclusions regarding the eyesight ability.

The finally derived result of the assessment of the eyesight acuity is indicated to the subject 20 in step S104.

FIGS. 2A-2C illustrate a second embodiment of the system for assessing eyesight acuity of the subject 20. In comparison to the first embodiment shown in FIGS. 1A and 1B, the second embodiment includes some alternative components as well as further refinements (additional components). Identical components are therein denoted by the same reference numerals as before.

The eye movement sensor 14′ according to the second embodiment comprises an electrooculographic (EOG) sensor 14′ instead of a camera 14. Such EOG sensors 14′ include a pair of electrodes or a plurality of electrodes which are attached to the head of the subject 20 either above and below the eyes or to the left and the right of the eyes. The EOG sensor 14′ monitors the eye movement by detecting a potential difference between said electrodes and assuming that the resting potential is constant, such that the recorded potential is a measure of the position of an eye of the subject 20. The result of the EOG measurement is again an eye movement signal 22 as exemplarily shown in FIG. 3. The EOG sensor 14′ is connected to the processing unit 16 either by means of a wireless connection or by means of a hard-wired connection.

A further difference to the first embodiment is the fact that the output unit 18′ according to the second embodiment includes a loudspeaker. The results of the assessment of the eyesight acuity may thus be fed back to the subject 20 in audible form. The loudspeaker 18′ also enables instructing the subject 20 by means of audible instructions. Such instructions may include instructions how to use the device 10, e.g. instructing the subject 20 how fast he/she should try to read and when he/she should start reading the text displayed on the display 12.

The system 10 according to the second embodiment furthermore comprises a proximity sensor 24. The proximity sensor 24 is configured to measure a distance between the display 12 and an eye/the eyes of the subject 20. The proximity sensor 24 may e.g. include an electromagnetic (e.g. infrared), photoelectric, radar-based or ultrasonic-based proximity sensor.

Still further, the device 10 according to the second embodiment includes the following additional components: A microphone 26 for recording the voice of the subject 20; an ambient light sensor 28 for measuring a brightness value of light in the ambience of the device 10; and a storage unit 30 for storing the result of an assessment of the eyesight acuity each time the eyesight acuity of the subject 20 is assessed by the processing unit 16. The latter-mentioned storage unit 30 may be either realized as a hard drive that is integrated in the device 10, or as an external storage unit, such as an external server, which is connected to the processing unit 16 via a data network.

The above-mentioned additional components of the system 10 according to the second embodiment enable the following improvements:

1. The processing unit 16 may assess the eyesight acuity of the subject 20 not only based on the above-mentioned analysis of the eye movement signal 22, but also by taking into account the distance between the display 12 and the eyes of the subject 20. The processing unit 16 may in this case particularly be configured to control the display 12 to vary at least one of a size, color and sharpness of the displayed text over time. Together this enables to monitor the reaction of the subject 20 onto a variation of the size, colour and/or sharpness of the displayed text. The system 10 may, for example, detect the type of eyesight disability of the subject 20 using the following indications: If the subject 20 reacts on a decrease of the displayed text by moving the display 12 closer to his/her eyes, the likelihood that the subject 20 suffers from myopia is increased. If the subject 20 reacts on a decrease of the displayed text by moving the display 12 farther away from his/her eyes, the likelihood that the subject 20 suffers from an astigmatism, hypermetropia or prespyopia is increased.

2. The possibility to store the results and parameters of former assessments of the eyesight acuity of the subject 20 in the storage unit 30 enables a comparison of different assessments taken over a longer time period. This specifically allows detecting trends of a deterioration of the subject's eyesight ability in very early phases.

3. The inclusion of a microphone 26 for recording the voice of the subject 20 enables an assessment mode in which the subject 20 reads out loud the displayed text and the processing unit 16 performs a speech recognition and a comparison of the speech recognition with the text that is actually displayed on the display 12. This increases the robustness of the assessment.

4. The provision of an ambient light sensor 28 allows accounting for the ambient conditions in the surrounding of the system 10. Based on the brightness value of the ambient light measured by the sensor 28, the subject 20 may receive a feedback via the loudspeaker 18 or via the display 12 inviting him to move to another place with more light before starting the assessment. Alternatively, the processing unit 16 may be configured to adjust at least of a size of the displayed text, a luminosity of the display 12, and a contrast of the display 12 based on the measured brightness value of the ambient light. According to a still further alternative, the processing unit 16 may incorporate the measured brightness value of the ambient light into the assessment of the eyesight acuity of the subject 20, e.g. by correcting the assessment results depending on the brightness level of the ambient light.

Despite the assessment of the eyesight ability, the system 10 may also support the subject 20 during regular usage. A long as the system 10 is not used for assessing the eyesight of the subject 20, the processing unit 16 may be configured to adjust at least one of (i) a size of the graphics and/or text displayed on the display 12, (ii) a luminosity of the display 12, and (iii) a contrast of the display 12 based on the analysis of the monitored eye movement. This may be particularly done when the system 10 is used for regular reading. The system 10 may then adjust the text size, the luminosity and/or the contrast of the display 12 to ease the reading process. As these parameters are adjusted, the ease of the reading may be assessed again to check whether the parameters have been adapted in the most optimal way given the disability of the subject 20. In other words, the system changes the settings in a personalized way for the subject 20.

FIG. 2C shows the same embodiment as illustrated in FIG. 2A with the difference that graphical symbols are displayed on the display 12 instead of text. These graphical symbols may for example, include circles with an opening at a specific position. Instead of reading a text, the subject 20 then has to determine at what position (left, right, up, down, upper left corner, lower right corner, etc.) the opening occurs in the different circles. the feedback of the subject 20 may again be recorded by means of the microphone 26 and evaluated by means of a speech recognition. The above-mentioned inclusion of the distance measurement of the proximity sensor 24, and the light measurement of the ambient light sensor 28 may be incorporated into the assessment in the same way as explained above.

It shall be noted that the components of the system 10 explained above with reference to the first embodiment shown in FIGS. 1A and 1B and the second embodiment shown in FIGS. 2A-2C may not only be combined in the above-mentioned ways, but may be permuted in an almost arbitrary manner without leaving the scope of the present invention as claimed in the appended claims.

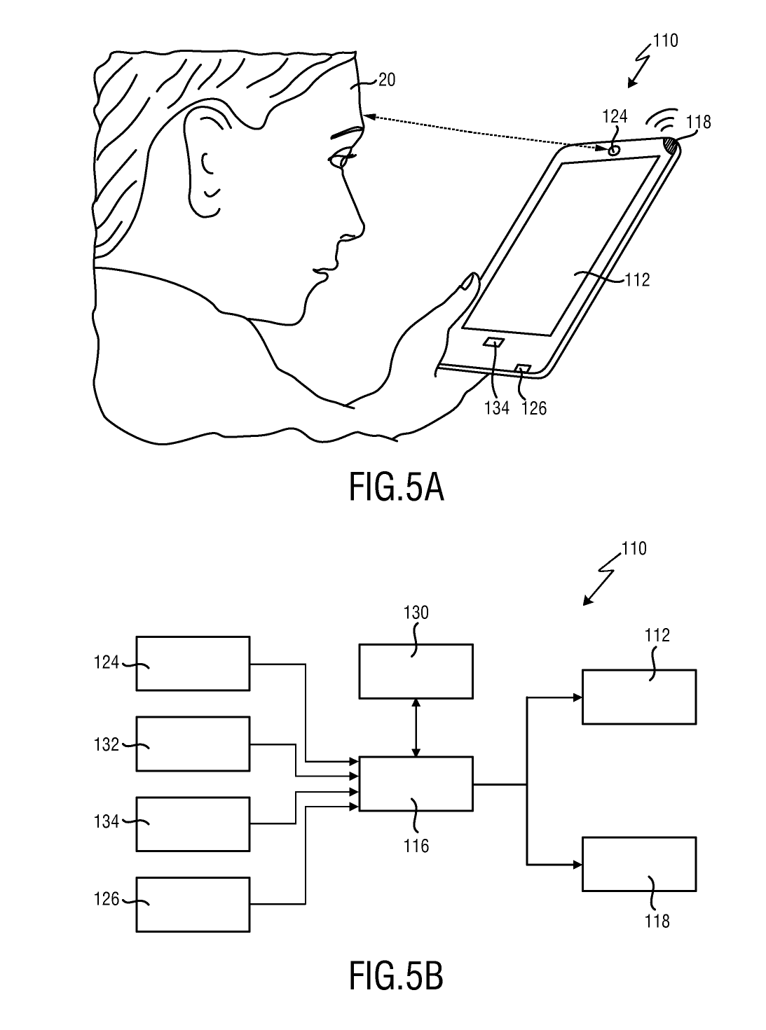

FIGS. 5A and 5B schematically illustrate a system for assessing hearing ability of the subject 20. Said system is denoted in its entirety by reference numeral 110.

The system 110 is in the presented example also included in a portable computing device, e.g. a tablet (similar as the system 10).

The system 110 for assessing hearing ability of the subject 20 includes an output unit 112 in the form of a display, a processing unit 116, a loudspeaker 118, a proximity sensor 124, a sound variation unit 132 and a feedback unit 134. Optionally, the system 110 may further comprise a microphone 126 and a storage unit 130. The loudspeaker 118 is configured to generate a sound. The sound variation unit 132 is configured to vary a frequency and/or loudness of the sound. This sound variation unit 132 may either be hardware-implemented or software-implemented. The sound variation unit 132 may, according to a first alternative, comprise a button with which the subject 20 may vary the frequency and/or loudness of the generated sound. This button does not have to be a physical button, but may also be a part of a touchscreen integrated into the display 112. According to a second alternative, the sound variation unit 132 may be a part of the processing unit 116 and configured to automatically vary the frequency and/or the loudness of the sound over time. The feedback unit 134 preferably comprises a user interface in the form of a button or a touchscreen integrated into the display 112 which enables the subject 20 to give the system 110 a feedback at what frequency and/or loudness he/she may recognize the generated sound. The proximity sensor 124 may be realized in a similar or even in the same manner as the proximity sensor 24 of the system 10 mentioned above. This proximity sensor 124 is configured to measure a distance between the loudspeaker 118 and the subject 20. The processing unit 116 may be realized as a microchip or another type of CPU (similar as the processing unit 16 of system 10).

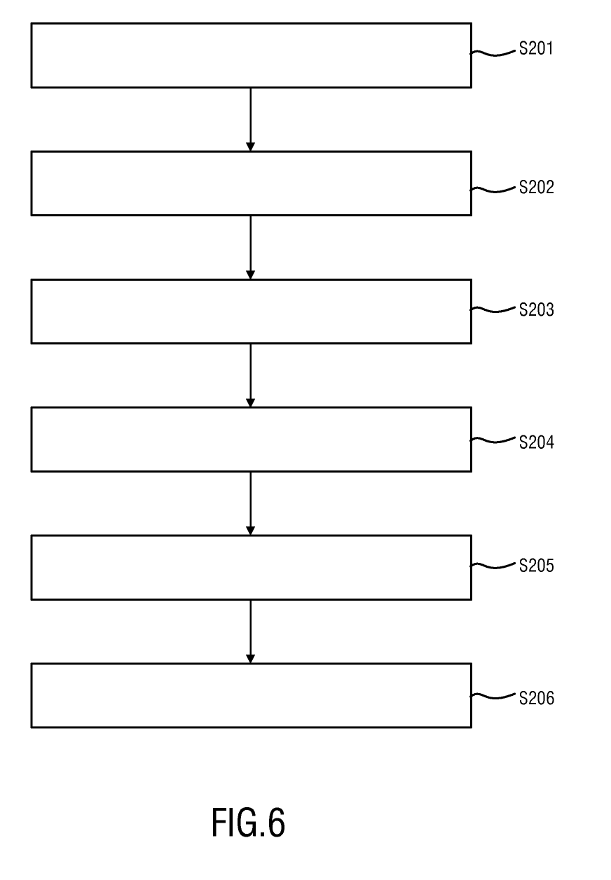

FIG. 6 schematically illustrates the method which is performed by the system 110. In a first step S201 the sound is generated via the loudspeaker 118. In step S202 this sound will be varied regarding its frequency and/or loudness by means of the sound variation unit 132. The loudness is, for example, increased gradually starting from silence and becoming louder and louder. The subject 20 then has to listen to the generated sound and give a feedback as soon as he/she recognizes the sound (step S203). This feedback may e.g. be given by means of touching the button 134 or tipping on the display 112. During the generation and variation of the sound, the proximity sensor 124 measures the distance between the loudspeaker 118 and the subject 20 (step S204). The subject 20 may additionally be given an instruction via the display 112 to keep the distance to the loudspeaker 118 constant during testing. In step S205 the processing unit may then assess the hearing ability of the subject 20 based on an analysis of the received feedback of the subject (i.e. the frequency and/or loudness the subject recognizes the generated sound the first time) and the distance between the loudspeaker 118 and the subject 20. Finally, the result of the assessment of the hearing ability may be indicated to the subject 20 via the display 112 in step S206.

The optional microphone 126 may be used to account for sounds in the ambience of the system 110. The processing unit 116 may be configured to react on the ambient sound level recorded by the microphone 126 in the following ways: According to a first alternative, the processing unit may generate a feedback to the subject 20 inviting the subject 20 to move to another place where the ambient sound level is lower. According to a second alternative, the processing unit 116 may be configured to adjust the frequency and/or loudness of the generated sound depending on the measured ambient sound level. According to a third alternative, the processing unit 116 may be configured to incorporate the measured ambient sound level into the assessment of the hearing ability of the subject, e.g. by subtracting the measured ambient sound level from the loudness at which the subject 20 indicated that he/she recognized the generated sound.

Similar as in the system 10, the storage unit 130 may be used to compare current assessments of the hearing ability of the subject 20 with former assessments stored in the storage unit 130 in order to derive a trend over time of the hearing ability of the subject 20.

It shall be noted that the system 10 for assessing the eyesight acuity of a subject 20 may also be combined with the system 110 for assessing the hearing ability of the subject 20 without leaving the scope of the present invention. Lastly, it shall be also noted that the herein presented methods in practice include a lot of concurrent method steps instead of sequential method steps and that FIGS. 4 and 6 simply show the method steps S101-S104 and S201-S206 only for simplicity reasons in a sequential order.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. A system for assessing eyesight acuity of a subject, comprising:
a display configured to display graphics and/or text to the subject;
an eye movement sensor configured to monitor an eye movement of the subject while the subject is watching the graphics and/or text displayed on the display;
a processing unit configured to assess the eyesight acuity of the subject based on an analysis of the monitored eye movement, wherein the analysis of the monitored eye movement includes an analysis of at least one of a duration of eye saccades, a frequency of eye saccades, a duration of eye fixations and a frequency of eye fixations in the monitored eye movement; and
an output unit configured to indicate the result of the assessment of the eyesight acuity.

2. (canceled)

3. (canceled)

4. A system as claimed in claim 1, wherein the processing unit is configured to control the display to vary at least one of a size, colour and sharpness of the graphics and/or the text over time, and wherein the processing unit is configured to assess the eyesight acuity of the subject based on the analysis of the monitored eye movement and the variation of the at least one of the size, colour and sharpness of the graphics and/or the text over time.

5. A system as claimed in claim 1, further comprising a proximity sensor for measuring a distance between the display and an eye of the subject, wherein the processing unit is configured to assess the eyesight acuity of the subject based on the analysis of the monitored eye movement and the distance between the display and the eye of the subject.

6. A system as claimed in claim 1, further comprising a microphone for recording a voice of the subject while reading out loud the graphics and/or the text displayed on the display, wherein the processing unit is configured to perform a speech recognition, and wherein the processing unit is configured to assess the eyesight acuity of the subject based on the analysis of the monitored eye movement and a comparison of the speech recognition with the graphics and/or the text displayed on the display.

7. A system as claimed in claim 1, wherein the processing unit is configured to adjust at least one of (i) a size of the graphics and/or text displayed on the display, (ii) a luminosity of the display, and (iii) a contrast of the display based on the analysis of the monitored eye movement.

8. A system as claimed in claim 1, further comprising an ambient light sensor for measuring a brightness value of light in the ambience of the system, wherein the processing unit is configured:
to generate a feedback regarding the measured brightness value of the ambient light, which feedback is indicated via the output unit; or
to adjust at least one of (i) a size of the graphics and/or text displayed on the display, (ii) a luminosity of the display, and (iii) a contrast of the display based on the measured brightness value of the ambient light, or
to incorporate the measured brightness value of the ambient light in the assessment of the eyesight acuity of the subject.

9. A system as claimed in claim 1, wherein the processing unit is configured to output instructions for the subject via the output unit, wherein said instructions support the subject using the system.

10. system as claimed in claim 1, further comprising a storage unit for storing the result of the assessment of the eyesight acuity each time the eyesight acuity of the subject is assessed by the processing unit, wherein the processing unit is configured to compare each new assessment with former assessments stored in the storage unit in order to derive a trend of the eyesight acuity of the subject over time.

11. (canceled)

12. A method for assessing eyesight acuity of a subject, comprising:
displaying graphics and/or text on a display;
monitoring an eye movement of the subject by means of an eye movement sensor while the subject is watching the graphics and/or text displayed on the display;
assessing the eyesight acuity of the subject based on an analysis of the monitored eye movement, wherein the analysis of the monitored eye movement includes an analysis of at least one of a duration of eye saccades, a frequency of eye saccades, a duration of eye fixations and a frequency of eye fixations in the monitored eye movement; and
indicating the result of the assessment of the eyesight acuity.

13. The computer program comprising program code means for causing a computer to carry out the steps of the method as claimed in claim 12 when said computer program is carried out on a computer.

14. A system for assessing hearing ability of a subject, comprising:
a loudspeaker for generating a sound;
a sound variation unit for varying a frequency and/or loudness of the sound;
a feedback unit for receiving feedback at what frequency and/or loudness the sound may be recognized by the subject;
a proximity sensor for measuring a distance between the loudspeaker and the subject;
a processing unit for assessing the hearing ability of the subject based on an analysis of the received feedback and the distance between the loudspeaker and the subject; and
an output unit for indicating the result of the assessment of the hearing ability.

15. A system as claimed in claim 14, wherein the sound variation unit comprises a user interface for manually varying the frequency and/or loudness of the sound and wherein the sound variation unit is configured to automatically vary the frequency and/or loudness of the sound over time.

16. (canceled)

17. A system as claimed in claim 14, further comprising a microphone for measuring a sound level in the ambience of the system, wherein the processing unit is configured:
to generate a feedback regarding the measured ambient sound level, which feedback is indicated via the output unit; or
to adjust the frequency and/or loudness of the generated sound based on the measured ambient sound level, or
to incorporate the measured ambient sound level in the assessment of the hearing ability of the subject.

18. A method for assessing hearing ability of a subject, comprising:
generating a sound by means of a loudspeaker;
varying a frequency and/or loudness of the sound;
receiving feedback at what frequency and/or loudness the sound may be recognized by the subject;
measuring a distance between the loudspeaker and the subject;
assessing the hearing ability of the subject based on an analysis of the received feedback and the distance between the loudspeaker and the subject; and
indicating the result of the assessment of the hearing ability.

19. The computer program comprising program code means for causing a computer to carry out the steps of the method as claimed in claim 18 when said computer program is carried out on a computer.
